(19) United States
(12) Patent Application Publication
(10) Pub. No.: US 2023/0233128 A1
(43) Pub. Date: Jul. 27, 2023

(54) Extracting Physiological Data from Raw Electrocardiography Data as Part of Magnetic Resonance Imaging

(71) Applicant: Siemens Healthcare GmbH, Erlangen (DE)

(72) Inventors: Ulrich Batzer, Spardorf (DE); Michael Roas-Löffler, Erlangen (DE); Christopher Horn, Erlangen (DE)

(73) Assignee: Siemens Healthcare GmbH, Erlangen (DE)

(21) Appl. No.: 18/102,232

(22) Filed: Jan. 27, 2023

(30) Foreign Application Priority Data
Jan. 27, 2022 (DE) 10 2022 200 926.6

(51) Int. Cl. A61B 5/308; A61B 5/00; A61B 5/055; A61B 5/346; A61B 5/0205

(52) U.S. Cl. CPC A61B 5/308; A61B 5/725; A61B 5/7278; A61B 5/055; A61B 5/346; A61B 5/0205

(57) ABSTRACT

In a method for extracting physiological data of an object under examination from ECG signals as part of MR imaging, raw ECG data comprising ECG signals may be captured from at least three electrodes located at different positions on an object under examination. The raw ECG data may be processed, which may include performing a first filtering using a first filter configured to extract an electrocardiogram, performing a second filtering using a second filter configured to identify a heartbeat, performing a third filtering using a third filter configured to extract and/or represent a respiratory movement, and/or performing a fourth filtering using a fourth filter configured to identify breathing. The processed raw ECG data including physiological data of the object under examination may be provided as an output.

### CROSS REFERENCE TO RELATED APPLICATIONS

This patent application claims priority to German Patent Application No. 102022200926.6, filed Jan. 27, 2022, which is incorporated herein by reference in its entirety.

### BACKGROUND

### Field

The disclosure relates to a method, to an electrocardiography (ECG) device, to a computer program product and to an electronically readable data storage medium for extracting physiological data from raw ECG data.

### Related Art

In a magnetic resonance device, a main magnet is normally used to apply a relatively high main magnetic field, for example of 1.5 or 3 or 7 Tesla, to the body to be examined of an object under examination, in particular of a patient. In addition, gradient pulses and radiofrequency pulses are used to induce radiofrequency signals in nuclear spins, which signals are received by suitable radiofrequency antennas, and reconstructed into image data. The timing of the gradient pulses and radiofrequency pulses is typically specified by MR control sequences. In MR imaging, MR control sequences are output, generating raw MR data, which can be reconstructed into MR image data. MR data can comprise raw MR data and/or MR image data. These MR control sequences can be synchronized with the heartbeat of the patient, which is advantageous in particular for cardiac examinations. For this purpose, an ECG device can be used to acquire an electrocardiogram, i.e. ECG data, from the patient before, or during, a magnetic resonance examination. This is typically done at a time at which the patient is located inside the magnetic resonance device and being exposed to the main magnetic field. In this situation, interactions occur between the main magnetic field, physical effects resulting therefrom and the ECG device, which interactions can affect the ECG signals.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 is a flowchart of a method according to an exemplary embodiment of the disclosure.

FIG. 2 is a flowchart of a method according to an exemplary embodiment of the disclosure.

FIG. 3 illustrates an ECG device according to an exemplary embodiment of the disclosure.

FIG. 4 illustrates an ECG device according to an exemplary embodiment of the disclosure.

FIG. 5 illustrates an ECG device according to an exemplary embodiment of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

The movement of the object under examination, in particular also caused by breathing, can affect the quality of the MR data and of the ECG data. Conventional methods for capturing a respiratory movement of the object under examination comprise chest straps and contactless methods, for instance based on camera monitoring and/or analyzing the MR data.

An object of the disclosure is to define a particularly efficient and robust method for capturing physiological data as part of ECG-monitored MR imaging.

The method according to the disclosure for extracting physiological data of an object under examination as part of MR imaging, in particular as part of ECG-monitored MR imaging, provides the following method steps:
capturing raw ECG data comprising ECG signals from at least three electrodes located at different positions on an object under examination;
processing the raw ECG data using at least one of the following methods:
first filtering by means of a first filter for extracting an electrocardiogram,
second filtering by means of a second filter for identifying a heartbeat,
third filtering by means of a third filter for extracting and/or representing a respiratory movement,
fourth filtering by means of a fourth filter for identifying breathing;
providing the processed raw ECG data comprising physiological data of the object under examination.

In the context of this method, the object under examination and the ECG device configured to capture the raw ECG data are typically arranged inside a patient placement region of a magnetic resonance device and/or exposed to a main magnetic field of a magnetic resonance device. The main magnetic field is typically a static main magnetic field of strength between 0.5 Tesla and 7 Tesla that is particularly homogeneous inside the patient placement region, in particular inside the examination region. The magnetic resonance device is typically configured to capture MR data; the ECG device is typically configured to capture raw ECG data of the object under examination. For this purpose, the ECG device is typically appropriately connected to the object under examination, typically by attached electrodes in the chest region of the object under examination.

Physiological data can comprise, for example, information relating to breathing, heartbeat, blood pressure, flow rate, functional MR data and/or an electrocardiogram. An electrode is typically configured to be arranged, in particular detachably fixed, on skin and/or a surface of an object under examination. An electrode may comprise a sensor configured to capture an electrical signal originating from the body, in particular originating from a cardiac movement and/or cardiac excitation of the object under examination. An electrical signal of this type can be referred to as an ECG signal. An electrode typically comprises an electrode attachment terminal, to which is attached an electrode lead, preferably one electrode lead. The connection between the electrode lead and the electrode can be detachable and/or permanent.

The electrode can be an adhesive electrode. The ECG device comprises at least three electrodes, preferably four electrodes. A first of the at least three electrodes may be located in a first position on the surface of the object under examination when capturing a first ECG signal. A second of the at least two electrodes may be located in a second position on the surface of the object under examination when capturing a second ECG signal. A third of the at least three electrodes may be located in a third position on the surface of the object under examination when capturing a third ECG signal. The first ECG signal, the second ECG signal and the third ECG signal may be captured simultaneously and/or during MR imaging of the object under examination. The first ECG signal, the second ECG signal and the third ECG signal can be referred to jointly as raw ECG data. In processing the raw ECG data, the stated methods can be applied separately to the individual ECG signals. The processing of the raw ECG data can comprise combining at least some of the individual ECG signals and/or applying the stated methods to the combined ECG signals.

Extracting an electrocardiogram and/or the first filtering can be carried out in particular on the basis of information relating to the object under examination and/or to a heart rate and/or to a reference value for an electrocardiogram and/or on the basis of results from the second filtering and/or from the third filtering and/or from the fourth filtering. Identifying a heartbeat and/or the second filtering can be carried out in particular on the basis of information relating to the object under examination and/or to an electrocardiogram and/or on the basis of results from the first filtering and/or from the third filtering and/or from the fourth filtering. An identified heartbeat in particular is a measure of the heart rate. Extracting and/or representing a respiratory movement and/or the third filtering may comprise determining a change in breathing over time, in particular a respiratory cycle, of the object under examination. The third filtering can be carried out on the basis of results from the second filtering and/or from the first filtering and/or from the fourth filtering. Identifying breathing and/or the fourth filtering may comprise detecting exhalation and/or inhalation. The fourth filtering can be carried out on the basis of results from the second filtering and/or from the first filtering and/or from the third filtering.

It is conventional to perform analog filtering of the raw ECG data to extract an electrocardiogram. It has been found that when the raw ECG data is processed according to the disclosure, in particular in combination with appropriate placement of the electrodes, i.e. given an appropriate choice of the first position, the second position and/or the third position, it is possible to determine a respiratory movement on the basis of the raw ECG data, which respiratory movement is superimposed on the ECG signals. It has been found in particular that the movement of a ribcage of the object under examination caused by breathing is sufficient to obtain a respiratory signal in the magnetic field of a magnetic resonance device having an ECG device. An ECG device comprises in addition to the electrodes typically electrode leads which connect the electrodes to a receiver, which electrode leads can form with the body a simple loop. Such a loop can be referred to as a coil of number of turns equal to one, in which, because of being located during the MR imaging in a magnetic field equal to the main magnetic field of the magnetic resonance device, is induced an electric current if the loop and/or the electrode lead is subject to a movement. An electric current can also be induced in the loop and/or the electrode lead when a temporary magnetic field gradient is generated by a gradient coil unit comprised by the magnetic resonance device as part of the imaging. This induced electric current is a measure of a respiratory movement. This induced electric current is typically captured as part of the ECG signals, where in particular it can lead to corruption of the electrocardiogram if there is no filtering and/or model-based data processing carried out.

This method makes it possible to ascertain physiological features, for instance the breathing of the object under examination, on the basis of ECG signals captured by a conventional ECG device and in particular without additional sensors and without further components. The breathing can thereby be captured particularly efficiently, robustly and cost-effectively.

According to an embodiment of the method, the processing of the raw ECG data is carried out without using a high-pass filter, in particular without an analog high-pass filter. Conventional ECG devices typically use an analog high-pass filter having a cutoff frequency of 1.5 Hz for example and/or use a digital low-pass filter. When the raw ECG data is processed accordingly by such a filter, respiratory movements are removed therefrom. This embodiment makes it possible to extract a respiratory movement from the raw ECG data.

According to an embodiment of the disclosure, the first filter is in the form of a bandpass filter for frequencies between 1.0 Hz and 40 Hz, and/or the second filter is in the form of a bandpass filter for frequencies between 0.1 Hz and 100 Hz, and/or the third filter is in the form of a bandpass filter for frequencies between 0.01 Hz and 2.0 Hz, and/or the fourth filter is in the form of a bandpass filter for frequencies between 0.01 Hz and 10.0 Hz.

The first filter can be in the form of a bandpass filter for frequencies between 1.0 Hz and 40 Hz, preferably between 1.3 Hz and 30 Hz, particularly preferably between 1.5 Hz and 25 Hz. Such a frequency range is particularly well suited to ascertaining an electrocardiogram of the object under examination, because typical electrical stimulations of the heart occur inside this frequency range. The second filter can be in the form of a bandpass filter for frequencies between 0.1 Hz and 100 Hz, preferably between 0.3 Hz and 60 Hz, particularly preferably between 0.5 Hz and 40 Hz. Such a frequency range is particularly well suited to detecting the heartbeat of the object under examination, because this is slightly higher than the frequency range of the first filter for ascertaining the electrocardiogram and thus is also suitable for detecting irregularities. The third filter can be in the form of a bandpass filter for frequencies between 0.01 Hz and 2.0 Hz, preferably between 0.03 Hz and 1.5 Hz, particularly preferably between 0.05 Hz and 1.0 Hz. Such a frequency range is particularly well suited to representing the breathing of the object under examination, because typical breathing occurs at this frequency. The fourth filter can be in the form of a bandpass filter for frequencies between 0.01 Hz and 10 Hz, preferably between 0.03 Hz and 6.0 Hz, particularly preferably between 0.05 Hz and 3.0 Hz. Such a frequency range is particularly well suited to detecting the breathing of the object under examination, because this is slightly higher than the frequency range of the third filter for representing the breathing and thus is also suitable for detecting high-frequency breathing and/or irregular breathing.

According to an embodiment of the method, the first filter and/or the second filter and/or the third filter and/or the fourth filter comprises a model-based filter, in particular a Kalman filter, and/or a non-linear filter, in particular a median filter. Such filters are particularly robust and universally deployable.

An embodiment of the method additionally comprises verifying the positioning of the at least three electrodes on the object under examination in accordance with the following method steps:
providing data comprising
   identified breathing, if fourth filtering of the raw ECG data is carried out, and/or
   identified heartbeat, if second filtering of the raw ECG data is carried out, and/or
   information relating to the object under examination, and/or
   a location of the object under examination relative to the magnetic resonance device, and/or
   system information on the magnetic resonance device;
ascertaining an optimum position for at least one electrode on the basis of the provided data and the processed raw ECG data;
providing the optimum position.

The information relating to the object under examination can comprise a size and/or a weight and/or MR image data, in particular three-dimensional MR image data, photographic image data and/or the indicators for thoracic breathing and/or abdominal breathing. This information can be based at least in part on MR data. The system information on the magnetic resonance device can comprise in particular information relating to a homogeneity and/or volume and/or strength of the main magnetic field. The location of the object under examination relative to the magnetic resonance device may comprise a position of an object under examination inside the main magnetic field. The location of the object under examination relative to the magnetic resonance device can be based on MR data.

The verification of the positioning, in particular of the initial positioning, of the at least three electrodes on the object under examination may additionally comprises ascertaining the current position of at least one electrode of the at least three electrodes. Ascertaining a deviation of the optimum position from the current position can be provided as an additional method step. In addition, information that the deviation is present and/or the deviation itself can be provided.

The optimum position of at least one electrode can be characterized by a desired and/or defined exposure with regard to the respiratory movement of the object under examination. In particular, the optimum position can be characterized by a maximum movement during breathing by the object under examination. The respiratory movement is typically superimposed on the ECG signals. The respiratory movement can affect the at least three ECG signals to varying degrees. This depends in particular on their initial positioning.

An optimum position may be ascertained for all of the at least three electrodes. The providing of the optimum position can comprise repositioning the at least one electrode from its initial position to the corresponding optimum positioning. The repositioning is typically performed manually by medical personnel.

In particular by taking account of the processed raw ECG data, this embodiment makes it possible to validate the quality of an identified breathing and/or a respiratory movement and/or the electrocardiogram and, in the event of poor quality, makes it possible to reposition the electrodes by providing the optimum position for the electrodes. This increases the quality of a respiratory cycle detected according to the disclosure and of an electrocardiogram detected according to the disclosure.

In an embodiment of the method, the providing comprises a visual representation of the optimum position, in particular a projection of the optimum position onto the object under examination. The visualization simplifies repositioning the electrodes to the optimum position.

An embodiment of the method additionally comprises repositioning at least one electrode of the at least three electrodes to the optimum position. Raw ECG data acquired after the repositioning has, according to this embodiment, a higher quality with regard to respiratory movement and/or electrocardiogram.

According to an embodiment of the method, the optimum position is characterized by a threshold value and/or a range for an amplitude of a respiratory movement of the object under examination and/or for an electrical stimulation resulting from a heartbeat of the object under examination.

The optimum position can be arranged in particular off-center, for instance on the side of the ribcage of the object under examination, in particular if a particularly high amplitude of a respiratory movement of the object under examination is desired. Equally, the optimum position can be characterized by a small amplitude of a respiratory movement of the object under examination. This can be desirable in particular for an ECG examination in the magnetic resonance device for which particularly high quality of the ECG data is desired, and/or the breathing is captured conventionally by a separate method such as a chest strap, for instance. In particular, the providing of the optimum position can be linked to a condition that provides that a threshold value for an amplitude of a respiratory movement of the object under examination is exceeded. This ensures that time-consuming correction is not performed for slight deviations from the optimum.

The disclosure also relates to an ECG device configured for use in combination with a magnetic resonance device and for performing a method according to the disclosure. The ECG device comprises at least three electrodes configured for arranging on an upper body of an object under examination, and comprises a receiver and at least three electrode leads that each connect one electrode of the at least three electrodes to the receiver, which receiver is configured to process the raw ECG data.

Each electrode lead is typically configured to connect one electrode to the receiver. The electrode lead typically comprises an electrically conducting cable that may be externally insulated. The electrode lead is typically configured to transmit to the receiver an ECG signal captured by the electrode connected to the electrode lead.

The receiver is configured to combine and/or to process and/or to analyze ECG signals captured by the at least three electrodes, and in particular to generate raw ECG data from the ECG signals. The receiver can comprise a filter configured to filter an ECG signal and/or can comprise an amplifier configured to amplify an ECG signal. The receiver can also comprise a status unit, which status unit is configured to identify the functionality and/or use of at least one electrode. The receiver can comprise a detector, which controls the ECG device centrally. The receiver can comprise a processor, which is configured to analyze at least ECG signals from a plurality of electrodes and/or to produce an ECG on the basis of these signals. The processor may be configured to process according to the disclosure raw ECG data and to extract physiological data from the raw ECG data.

For this purpose, the receiver typically has an input, a processor and an output. The captured raw ECG data and/or data for verifying the positioning of the at least three electrodes on the object under examination can be provided to the processor via the input. Further functions, algorithms or parameters needed in the method can be provided to the processor via the input. The processed raw ECG data and/or further results from an embodiment of the method according to the disclosure can be provided via the output. The processor and/or the receiver can be integrated in the ECG device. The processor and/or the receiver can also be installed separately from the ECG device. The processor and/or the receiver can be connected to the ECG device.

The electrode leads, which are connected to the body via electrodes, can form with the object under examination a simple electrical loop. Such a loop can be referred to as a coil of number of turns equal to one. If the object under examination is located in a magnetic resonance device for MR imaging and/or for preparing the MR imaging, then the electrode leads, and hence the simple electrical loop, are exposed to the main magnetic field of the magnetic resonance device. During a movement, an electric current is then induced in the simple electrical loop. This induced electric current is a measure of a movement of the electrode leads. The induced electric current correlates with the respiratory movement because the electrode leads are typically positioned on the upper body of the object under examination.

An embodiment of the ECG device additionally comprises at least two coil units, each configured to capture a movement signal representing a movement to which the particular coil unit of the at least two coil units is subject, wherein at least one coil unit of the at least two coil units is arranged on an electrode of the at least three electrodes and/or on an electrode lead of the at least three electrode leads and/or on the receiver, and comprises a movement detector, which movement detector is configured to extract a respiratory movement on the basis of the movement signals.

In addition to and/or instead of the simple electrical loop formed from the electrode leads at least two coil units having the same operating principle can be used. Each of the at least two coil units is exposed to a main magnetic field during the examination of the object under examination by the magnetic resonance device when the object under examination is located inside the patient placement region of the magnetic resonance device. If a coil unit of the at least two coil units is subject to a movement, an electric current is induced therein that is a measure of the movement of the coil unit and can correspond to the movement signal. A movement signal is typically time dependent. Consequently, each coil unit of the at least two coil units is configured to measure a change in its position over time. In the case that a coil unit of the at least two coil units is arranged on an electrode of the at least three electrodes and/or on an electrode lead of the at least three electrode leads and/or on the receiver, the coil unit typically has a physical connection to each component concerned and can capture a movement of the component concerned. If the coil unit is arranged on a part of the ECG device, which part is moved by the breathing of the object under examination, the respiratory signal is imposed on the coil unit. The ECG device can also comprise only one coil unit configured to capture a movement signal.

The receiver can comprise the movement detector. The movement detector can be configured to process in a consolidated manner the movement signals captured by the at least two coil units, in order to extract an averaged respiratory movement. The movement detector can be configured to process separately the movement signals captured by the at least two coil units, in order to exploit a spatial distribution of the coil units and/or to extract a spatially resolved respiratory movement.

This embodiment of the ECG device allows spatial detection of breathing for the same amount of preparation effort as with a conventional ECG device.

In addition, the ECG device allows extraction of the breathing and/or heartbeat. The at least two coil units allow spatial resolution of the breathing and in particular differentiation between abdominal and thoracic breathing. This makes prospective 3D motion-correction possible.

According to an embodiment of the ECG device, in the case that the ECG device is arranged on the upper body of the object under examination, the at least two coil units are distributed over a surface of the upper body in such a way that they are configured to capture abdominal breathing and thoracic breathing.

The at least two coil units may comprise at least five coil units, which are configured to be arranged on a ribcage and on an abdomen of the object under examination. The at least five coil units may be spatially distributed evenly on the object under examination. This allows good differentiation of the respiratory movement and particularly precise extraction of the breathing.

According to an embodiment of the ECG device, at least one of the at least two coil units is a separate coil unit, which separate coil unit is not in direct contact with an electrode of the at least three electrodes and/or is not in direct contact with an electrode lead of the at least three electrode leads and/or is not in direct contact with the receiver.

The separate coil unit can be positioned individually on an upper body of the object under examination. In particular, by appropriate positioning of the separate coil unit on a body part moved by the breathing, the respiratory signal from this body part can be imposed on the separate coil unit. This makes it possible to capture a respiratory movement independently of the electrodes and/or the electrode leads and/or the receiver of the ECG device, allowing particularly precise capture of the respiratory movement and the ECG.

According to an embodiment of the ECG device, the at least two coil units comprise thermal insulation and/or a housing unit. The thermal insulation reduces the influence of temperature on the coil unit, in particular actuated by heating by the object under examination, and thus allows more precise measurement of the movement. A housing unit can protect a coil unit from external mechanical influences, in particular from damage.

According to an embodiment of the ECG device, at least two coil units of the at least two coil units are connected to each other. The at least two coil units may be movably connected to each other and/or form an array of coil units. This allows extraction of a three-dimensional respiratory movement. Arranging said at least two interconnected coil units parallel to the receiver and/or with cutting edge inside the receiver can be particularly sensitive.

According to an embodiment of the ECG device, at least one of the at least two coil units comprises an electrical coil of circumference between 2 cm and 30 cm and/or a number of turns between 1 and 10.

According to this embodiment, at least one of the at least two coil units comprises an electrical coil of circumference between 2 cm and 30 cm, preferably between 4 cm and 25 cm, particularly preferably between 7 cm and 20 cm. According to this embodiment, at least one of the at least two coil units has a number of turns of at most 12, preferably of at most 10, particularly preferably of at most 5. The number of turns and/or the circumferences of the electrical coils of the at least two coil units can differ from each other or be the same. Such circumferences and number of turns can be integrated well in electrodes, electrode leads and/or receivers. In addition, such coil units have a good sensitivity with regard to movement in main magnetic fields between 0.2 Tesla and 7 Tesla, in particular between 0.5 Tesla and 3 Tesla.

According to an embodiment of the ECG device, the movement detector comprises an analog filter and/or an amplifier and/or an analog-to-digital converter (ADC). The movement detector may be configured for digital signal processing.

The movement detector is configured to extract a single averaged respiratory movement simultaneously and in a consolidated manner from the movements captured by the at least two coil units. The movement detector can be configured to extract a spatially resolved respiratory movement. Such a movement detector is particularly versatile.

According to an embodiment of the ECG device, the movement detector comprises an analog filter in the form of a low-pass filter up to 150 Hz, preferably up to 100 Hz, and/or an amplifier having a gain of between 6 and 12, preferably between 8 and 10, and/or an analog- to-digital converter (ADC) having an operating range between 100 Hz and 300 Hz, preferably between 150 Hz and 250 Hz, and/or a resolution of less than 15 μV per least significant bit (μV/LSB), preferably less than 10 μV/LSB. The low-pass filter makes it possible to confine the captured movement signal efficiently to potentially breathing-induced movement, and reduces higher-frequency influences on the movement signal that cannot be induced by respiratory movement. The amplifier makes it possible to amplify the captured movement signals, in particular according to the strength of the main magnetic field of the magnetic resonance device, which correlates with the strength of the movement signals. A gain between 6 and 12, preferably between 8 and 10, allows efficient and appropriate amplification in this case.

Further embodiments of the ECG device according to the disclosure are similar in design to the embodiments of the method according to the disclosure. The ECG device can comprise further control components that are needed and/or advantageous for performing a method according to the disclosure. The ECG device can also be configured to send control signals and/or to receive and/or to process control signals in order to perform a method according to the disclosure. Computer programs and further software, by means of which the processor of the receiver and/or of the movement detector automatically controls and/or executes a method sequence of a method according to the disclosure, can be stored on a memory of the receiver and/or movement detector.

A computer program product according to the disclosure can be loaded directly into a memory of a programmable receiver and/or movement detector, and comprises program code means in order to perform a method according to the disclosure when the computer program product is executed in the receiver and/or movement detector. The method according to the disclosure can thereby be performed quickly, reproducibly and robustly. The computer program product is configured such that it can perform the method steps according to the disclosure by means of the receiver and/or movement detector. The movement detector can be integrated in the receiver. The movement detector can be equivalent to the processor. The receiver and/or movement detector must each have the necessary specification such as, for example, an appropriate RAM, an appropriate graphics card or an appropriate logic unit, in order to be able to perform the respective method steps efficiently. The computer program product is stored, for example, on an electronically readable medium or on a network or server, from where it can be loaded into the processor of a local receiver and/or movement detector, which processor may have a direct connection to the ECG device or may form part of the ECG device. In addition, control data of the computer program product can be stored on an electronically readable data storage medium. The control data in the electronically readable data storage medium can be configured such that it performs a method according to the disclosure when the data storage medium is used in a receiver and/or movement detector of an ECG device. Examples of electronically readable data storage media are a DVD, a magnetic tape or a USB stick, on which is stored electronically readable control data, in particular software. When this control data (software) is read from the data storage medium and stored in a receiver and/or movement detector of an ECG device, all the embodiments according to the disclosure of the above-described method can be performed.

The disclosure is also based on an electronically readable data storage medium, on which is stored a program that is intended to perform a method for extracting physiological data of an object under examination from ECG signals as part of MR imaging.

The advantages of the ECG device according to the disclosure, of the computer program product according to the disclosure and of the electronically readable data storage medium according to the disclosure are essentially the same as the advantages of the method according to the disclosure for extracting physiological data of an object under examination from ECG signals as part of MR imaging, which are presented in detail above. Features, advantages or alternative embodiments mentioned in this connection can also be applied to the other claimed subject matter, and vice versa.

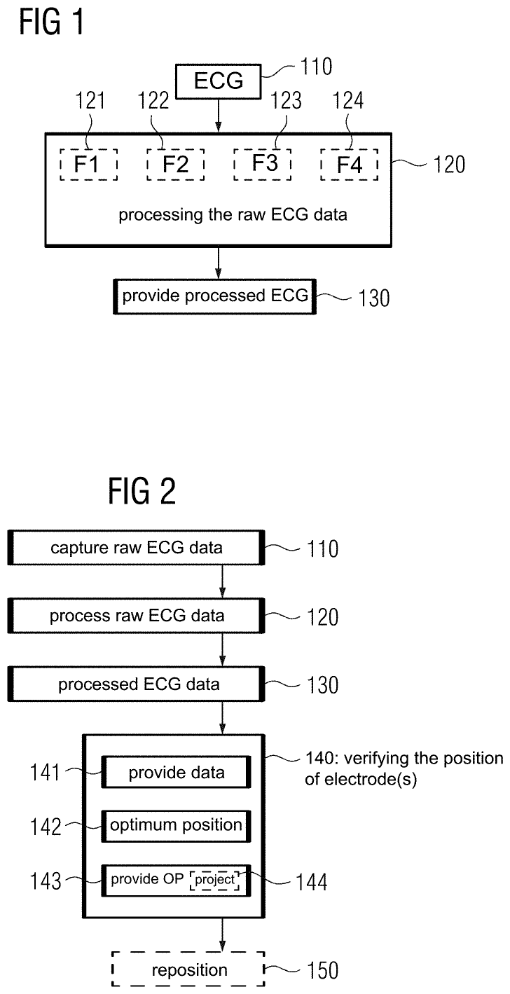

FIG. 1 shows a flow diagram of a first embodiment of a method according to the disclosure for extracting physiological data of an object under examination from ECG signals as part of MR imaging. The method comprises, in method step 110, capturing raw ECG data comprising ECG signals from at least three electrodes located at different positions on an object under examination. In method step 120, the raw ECG data is processed using at least one of the following methods. Method 121 comprises first filtering by means of a first filter for extracting an electrocardiogram. Method 122 comprises second filtering by means of a second filter for identifying a heartbeat. Method 123 comprises third filtering by means of a third filter for extracting and/or representing a respiratory movement. Method 124 comprises fourth filtering by means of a fourth filter for identifying breathing. In the final method step 130, the processed raw ECG data comprising physiological data of the object under examination is provided.

FIG. 2 shows a flow diagram of a second embodiment of a method according to the disclosure. In comparison with the first embodiment shown in FIG. 1, the method additionally comprises, in method step 140, verifying the positioning of the at least three electrodes on the object under examination in accordance with the following method steps:

In an exemplary embodiment, method step 141 comprises providing data comprising:
identified breathing, if fourth filtering of the raw ECG data is carried out, and/or
identified heartbeat, if second filtering of the raw ECG data is carried out, and/or
information relating to the object under examination, and/or
a location of the object under examination relative to the magnetic resonance device, and/or
system information on the magnetic resonance device.

In method step 142, an optimum position for at least one electrode is ascertained on the basis of the provided data. Method step 143 comprises providing the optimum position, which can comprise, in method step 144, projecting the optimum position onto the object under examination. The optional method step 150 comprises repositioning at least one electrode of the at least three electrodes to the optimum position.

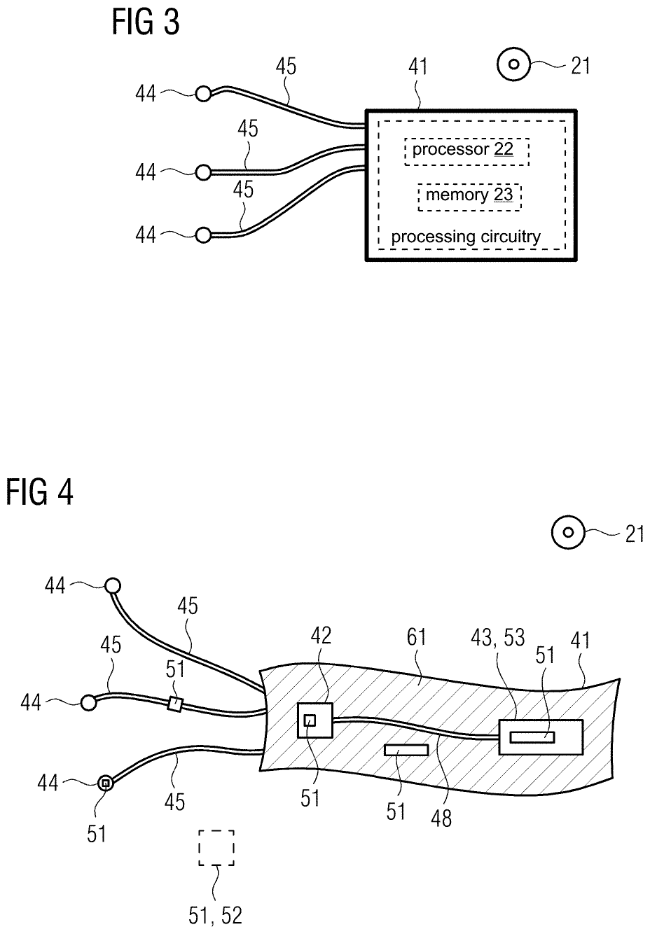

FIG. 3 shows a schematic diagram of a first embodiment of an ECG device according to the disclosure. The ECG device comprises at least three electrodes 44 configured to be arranged on the upper body of an object under examination, and comprises a receiver 41. Each electrode 44 of the at least three electrodes 44 is configured to capture an ECG signal. The at least three electrodes 44 are typically jointly configured to capture the raw ECG data. In addition, the ECG device comprises at least three electrode leads 45 that each connect one electrode 44 of the at least three electrodes 44 to the receiver 41. The ECG is configured, in particular together with the receiver 41 comprising a processor, to perform a method according to the disclosure for processing the raw ECG data, in particular for extracting physiological data on the basis of raw ECG data.

For this purpose, the receiver 41, in particular a processor 22, which is comprised by the receiver 41, comprises computer programs and/or software, which can be loaded directly into a memory 23 of the receiver 41, and has program means in order to perform a method for capturing breathing when the computer programs and/or software are executed in the processor 22. The receiver 41 may include processing circuitry that is configured to perform one or more functions and/or operations of the receiver 41, which may include executing the computer programs and/or software. In this example, the processing circuitry may include the processor 22 and/or memory 23. Alternatively, or additionally, the computer programs and/or software may be stored on an electronically readable data storage medium (memory) 21, which is embodied separately from the receiver 41, wherein data access to the electronically readable data storage medium 21 can be made by the receiver 41 via a data network.

A method for extracting physiological data can also exist in the form of a computer program product, which implements the method in the receiver 41 and/or the processor 22 when it is executed therein. Equally, there can be an electronically readable data storage medium 21 comprising electronically readable control data stored thereon, which data comprises at least one such computer program product as just described, and is configured such that it performs the described method when the electronically readable data storage medium 21 is used in a receiver 41 and/or a processor 22 of an ECG device. The receiver 41 may have an output, via which the physiological data of the object under examination can be provided.

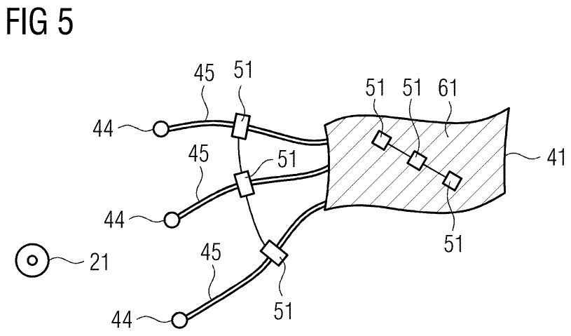

FIG. 4 shows a schematic diagram of a second embodiment of an ECG device according to the disclosure. In comparison with the first embodiment, this additionally comprises at least two coil units 51, 52 which are each configured to capture a movement to which the particular coil unit 51 of the at least two coil units 51, 52 is subject. At least one coil unit 51 of the at least two coil units 51, 52 is arranged on an electrode 44 of the at least three electrodes 44 and/or on an electrode lead 45 of the at least three electrode leads 45 and/or on the receiver 41. The receiver 41 may include an amplifier 42 and a detector 43, which are connected to a cable unit 48. The amplifier 42, the detector 43, and/or the cable unit 48 are may be embedded in a positioning unit 61 and/or enclosed at least partially thereby. The positioning unit 61 may comprises flexible and/or soft material. The amplifier 42 is configured to amplify the ECG signals. In an exemplary embodiment, the amplifier 42 and/or detector 43 may include processing circuitry that is configured to perform their respective function(s).

The ECG device according to the second embodiment additionally comprises a movement detector 53. Each of the at least two coil units 51, 52 may be connected to the movement detector 53, in particular in order to transmit the captured movement.

The movement detector 53 is configured to capture and/or extract a respiratory movement and/or breathing on the basis of the movements captured by the at least two coil units 51, 52.

For this purpose, the movement detector 53 has computer programs and/or software, which can be loaded directly into a memory (not presented in greater detail) of the movement detector 53 and has program means in order to perform a method for capturing breathing when the computer programs and/or software are executed in the movement detector 53. The movement detector 53 has for this purpose a processor (e.g. processor 22, not presented in greater detail), which is configured to execute the computer programs and/or software. Alternatively, the computer programs and/or software can also be stored on an electronically readable data storage medium 21, which is embodied separately from the movement detector 53, wherein data access to the electronically readable data storage medium 21 can be made by the movement detector 53 via a data network.

A method for extracting physiological data can also exist in the form of a computer program product, which implements the method in the movement detector 53 when it is executed in the movement detector 53. Likewise, there can also be an electronically readable data storage medium 21 comprising electronically readable control data stored thereon, which data comprises at least one such computer program product as just described, and is configured such that it performs the described method when the electronically readable data storage medium 21 is used in a movement detector 53 of an ECG device.

The movement detector 53 can be integrated in the receiver 41 and/or the detector 43 and/or in the processor, and/or can be comprised thereby. The processor can be integrated in the receiver 41 and/or the detector 43, and/or can be comprised thereby. The movement detector 53 can comprise an analog filter (not presented in greater detail) and/or an amplifier and/or an ADC.

Optionally, at least one of the at least two coil units 51, 52 is a separate coil unit 52, which separate coil unit 52 is not in direct contact with an electrode 44 of the at least three electrodes 44 and/or is not in direct contact with an electrode lead 45 of the at least three electrode leads 45 and/or is not in direct contact with the receiver 41. Optionally, at least one of the at least two coil units 51, 52 can comprise thermal insulation (not presented in greater detail) and/or a housing unit.

FIG. 5 shows a schematic diagram of a third embodiment of an ECG device according to the disclosure. This embodiment differs from the second embodiment of the ECG device according to the disclosure in that three coil units 51 are connected to each other. In particular, the three coil units 51 form an array, which is arranged across different electrode leads 45 and/or with a cutting edge inside the receiver 41.

Although the disclosure has been illustrated and described in detail using the preferred exemplary embodiments, the disclosure is not limited by the disclosed examples, and a person skilled in the art can derive other variations therefrom without departing from the scope of protection of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for extracting physiological data of an object under examination from electrocardiography (ECG) signals as part of magnetic resonance (MR) imaging, comprising:
capturing raw ECG data comprising ECG signals, using an ECG device, from at least three electrodes located at different positions on an object under examination;
processing the raw ECG data, the processing including:
performing a first filtering using a first filter configured to extract an electrocardiogram,
performing a second filtering using a second filter configured to identify a heartbeat,
performing a third filtering using a third filter configured to extract and/or represent a respiratory movement, and/or
performing a fourth filtering using a fourth filter configured to identify breathing; and
providing an electronic data signal, representing the processed raw ECG data including physiological data of the object under examination, as an output of the ECG device.

2. The method as claimed in claim 1, wherein the processing of the raw ECG data uses a high-pass filter.

3. The method as claimed in claim 1, wherein:
the first filter is a bandpass filter configured for frequencies between 1.0 Hz and 40 Hz;
the second filter is a bandpass filter configured for frequencies between 0.1 Hz and 100 Hz;
the third filter is a bandpass filter configured for frequencies between 0.01 Hz and 2.0 Hz; and/or
the fourth filter is a bandpass filter configured for frequencies between 0.01 Hz and 10.0 Hz.

4. The method as claimed in claim 1, wherein the first filter, the second filter, the third filter, and/or the fourth filter comprises a model-based filter and/or a non-linear filter.

5. The method as claimed in claim 1, further comprising: verifying a positioning of the at least three electrodes on the object under examination, the verifying including:
providing data comprising:
identified breathing in response to the fourth filtering of the raw ECG data being performed,
identified heartbeat in response to the second filtering of the raw ECG data being performed,
information relating to the object under examination,
a location of the object under examination relative to the magnetic resonance device, and/or
system information on the magnetic resonance device;
ascertaining an optimum position for one or more of the at least three electrodes based on the provided data and the processed raw ECG data; and
providing the optimum position.

6. The method as claimed in claim 5, wherein the providing the optimum position comprises projecting the optimum position onto the object under examination.

7. The method as claimed in claim 5, further comprising repositioning one or more of the at least three electrodes to the optimum position.

8. The method as claimed in claim 5, wherein the optimum position is a threshold value and/or a range for:
an amplitude of a respiratory movement of the object under examination, and/or
an electrical stimulation resulting from a heartbeat of the object under examination.

9. A non-transitory computer-readable storage medium with an executable program stored thereon, that when executed, instructs a processor to perform the method of claim 1.

10. An electrocardiography (ECG) device configured for use in combination with a magnetic resonance (MR) device, comprising:
at least three electrodes arrangeable on an object under examination and configured to capture ECG signals from the object under examination;
a receiver including at least three electrode leads that respectively connect the at least three electrodes to the receiver, the receiver being configured to:
capture ECG data based on the ECG signals;
process the ECG data to generate processed ECG data including physiological data of the object under examination; and
provide an electronic data signal representing the processed raw ECG data as an output of the receiver.

11. The ECG device as claimed in claim 10, comprising:
at least two coil units, each being configured to capture a movement signal representing a movement to which the respective coil unit is subject, wherein:
at least one of the at least two coil units is arranged on: an electrode of the at least three electrodes, an electrode lead of the at least three electrode leads, and/or the receiver; and
a movement detector configured to extract a respiratory movement based on at least one of the movement signals.

12. The ECG device as claimed in claim 11, wherein, in a case where the ECG device is arranged on an upper body of the object under examination, the at least two coil units are distributed over a surface of the upper body such that the at least two coil units are configured to capture abdominal breathing and thoracic breathing.

13. The ECG device as claimed in claim 11, wherein at least one of the at least two coil units is a separate coil unit that is:
not in direct contact with an electrode of the at least three electrodes;
not in direct contact with an electrode lead of the at least three electrode leads; and/or
not in direct contact with the receiver.

14. The ECG device as claimed in claim 11, wherein the at least two coil units comprise thermal insulation and/or a housing unit.

15. The ECG device as claimed in claim 11, wherein at least two coil units of the at least two coil units are connected to each other.

16. The ECG device as claimed in claim 11, wherein at least one of the at least two coil units comprises an electrical coil of circumference between 2 cm and 30 cm and/or a number of turns between 1 and 10.

17. The ECG device as claimed in claim 11, wherein the movement detector comprises: an analog filter, an amplifier, and/or an analog-to-digital converter (ADC).

18. The ECG device as claimed in claim 11, wherein the movement detector comprises:
an analog low-pass filter up to 150 Hz,
an amplifier having a gain of between 6 and 12, and/or
an analog-to-digital converter (ADC) having an operating range between 100 Hz and 300 Hz and/or a resolution of less than 15 μV per least significant bit (μV/LSB).

19. The ECG device as claimed in claim 10, wherein the processing of the ECG data comprises filtering the ECG data to: extract an electrocardiogram, identify a heartbeat, extract and/or represent a respiratory movement, and/or identify breathing.
